(19) United States
(12) Patent Application Publication
(10) Pub. No.: US 2022/0378378 A1
(43) Pub. Date: Dec. 1, 2022

(54) APPARATUS AND METHOD FOR PREDICTING CARDIOVASCULAR RISK FACTOR

(71) Applicants: SAMSUNG SDS CO., LTD., Seoul (KR); SAMSUNG MEDICAL CENTER, Seoul (KR)

(72) Inventors: Soo Ah CHO, Seoul (KR); Joon Ho LEE, Seoul (KR); Joon Seok LEE, Seoul (KR); Ji Eun SONG, Seoul (KR); Min Young LEE, Seoul (KR); Su Jeong SONG, Seoul (KR)

(21) Appl. No.: 17/637,564

(22) PCT Filed: Aug. 21, 2020
(86) PCT No.: PCT/KR2020/011163

§ 371 Date: Aug. 8, 2022

(30) Foreign Application Priority Data
Aug. 23, 2019 (KR) 10-2019-0103930

(51) Int. Cl. A61B 5/00; A61B 3/12; A61B 5/02; G16H 50/30; G16H 50/20

(52) U.S. Cl. CPC A61B 5/7275; A61B 3/12; A61B 5/7267; A61B 5/02007; G16H 50/30; G16H 50/20; A61B 2576/02

(57) ABSTRACT

An apparatus for predicting a cardiovascular risk factor according to an embodiment includes a target cardiovascular risk factor predicting module for producing an initial prediction value for a target cardiovascular risk factor from a fundus image, at least one related cardiovascular risk factor predicting module for producing respective prediction values for at least one related cardiovascular risk factor from the fundus image, and a combining module for producing a final prediction value for the target cardiovascular risk factor on the basis of the initial prediction value for the target cardiovascular risk factor and the respective prediction values for the at least one related cardiovascular risk factor.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/011163 filed on Aug. 21, 2020, which claims priority to the benefit of Korean Patent Application No. 10-2019-0103930 filed in the Korean Intellectual Property Office on Aug. 23, 2019, the entire contents of which are incorporated herein by reference.

### BACKGROUND

### 1. Technical Field

The present disclosure relates to a cardiovascular risk factor prediction technology.

### 2. Background Art

Cardiovascular disease may be a major disease leading to death, and in order to suggest appropriate treatment to the patient, the risk of heart attack, myocardial infarction, or the like should be confirmed based on the values of various cardiovascular risk factors.

Some of the cardiovascular risk factors may be measured with a simple examination such as a blood test, but some cardiovascular risk factors such as a coronary artery calcification score may be measured by a relatively high-cost test or a test that is burdensome, such as exposure to radiation or the like.

Meanwhile, a fundus image may be a means to non-invasively observe blood vessels in detail, and may be useful for eye disease screening due to low examination cost thereof. However, there may be problems in that the method for predicting a cardiovascular disease, based on an existing fundus image, may not only require a separate detailed examination for patients at high risk of major cardiovascular disease, but also reduce the prediction accuracy when electronic health record (EHR) information is missing.

### SUMMARY

An aspect of the present disclosure is to provide an apparatus and a method for predicting a cardiovascular risk factor.

An apparatus for predicting a cardiovascular risk factor, according to an embodiment of the present disclosure, includes a target cardiovascular risk factor predicting module producing an initial prediction value for a target cardiovascular risk factor from a fundus image; at least one related cardiovascular risk factor predicting module producing a prediction value for each of at least one related cardiovascular risk factor from the fundus image; and a combining module producing a final prediction value for the target cardiovascular risk factor, based on the initial prediction value for the target cardiovascular risk factor and the prediction value for each of the at least one related cardiovascular risk factor.

The target cardiovascular risk factor predicting module may produce the initial prediction value using a target cardiovascular risk factor prediction model, pre-trained using a plurality of pre-collected fundus images and an actually measured value for the target cardiovascular risk factor corresponding to each of the plurality of pre-collected fundus images.

The target cardiovascular risk factor prediction model may be a convolutional neural network (CNN)-based prediction model.

The at least one related cardiovascular risk factor predicting module may produce the prediction value using a related cardiovascular risk factor prediction model, pre-trained using a plurality of pre-collected fundus images and an actually measured value for each of the at least one related cardiovascular risk factor corresponding to each of the plurality of pre-collected fundus images.

The related cardiovascular risk factor prediction model may be a convolutional neural network (CNN)-based prediction model.

The combining module may produce the final prediction value using a prediction result binding model, pre-trained using an initial prediction value for the target cardiovascular risk factor, a prediction value for each of the at least one related cardiovascular risk factor, and an actually measured value for the target cardiovascular risk factor, with regard to each of a plurality of pre-collected fundus images.

The prediction result binding model may be a prediction model based on one of a regression analysis or an artificial neural network.

The target cardiovascular risk factor may be a coronary artery calcification score (CACS).

The target cardiovascular risk factor may be a carotid artery intima thickness.

The at least one related cardiovascular risk factor may include at least one of age, sex, smoking, a glycosylated hemoglobin level, blood pressure, a pulse wave, blood sugar level, cholesterol level, creatinine level, insulin level, or intraocular pressure.

A method for predicting a cardiovascular risk factor, according to an embodiment of the present disclosure, includes producing an initial prediction value for a target cardiovascular risk factor from a fundus image; producing a prediction value for each of at least one related cardiovascular risk factor from the fundus image; and producing a final prediction value for the target cardiovascular risk factor, based on the initial prediction value for the target cardiovascular risk factor and the prediction value for each of the at least one related cardiovascular risk factor.

The producing an initial prediction value may produce the initial prediction value using a target cardiovascular risk factor prediction model, pre-trained using a plurality of pre-collected fundus images and an actually measured value for the target cardiovascular risk factor corresponding to each of the plurality of pre-collected fundus images.

The target cardiovascular risk factor prediction model may be a convolutional neural network (CNN)-based prediction model.

The producing a prediction value for each of at least one related cardiovascular risk factor may produce the prediction value using a related cardiovascular risk factor prediction model, pre-trained using a plurality of pre-collected fundus images and an actually measured value for each of the at least one related cardiovascular risk factor corresponding to each of the plurality of pre-collected fundus images.

The related cardiovascular risk factor prediction model may be a convolutional neural network (CNN)-based prediction model.

The producing a final prediction value may produce the final prediction value using a prediction result binding model, pre-trained using an initial prediction value for the target cardiovascular risk factor, a prediction value for each of the at least one related cardiovascular risk factor, and an actually measured value for the target cardiovascular risk factor, with regard to each of a plurality of pre-collected fundus images.

The prediction result binding model may be a prediction model based on one of a regression analysis or an artificial neural network.

The target cardiovascular risk factor may be a coronary artery calcification score (CACS).

The target cardiovascular risk factor may be a carotid artery intima thickness.

The at least one related cardiovascular risk factor may include at least one of age, sex, smoking, a glycosylated hemoglobin level, blood pressure, a pulse wave, blood sugar level, cholesterol level, creatinine level, insulin level, or intraocular pressure.

According to embodiments of the present disclosure, prediction of a cardiovascular risk factor may be performed using only a fundus image, to predict the cardiovascular risk factor at low cost and without burden such as exposure to radiation.

In addition, according to embodiments of the present disclosure, predictive performance may be improved by using a prediction value of a related cardiovascular risk factor, in addition to a target cardiovascular risk factor to be predicted, to maintain prediction accuracy even without separate electronic health record (EHR) information.

### BRIEF DESCRIPTION OF THE DRAWINGS

FIG. 1 is a block diagram of an apparatus for predicting a cardiovascular risk factor according to an embodiment of the present disclosure.

FIG. 2 is a flowchart of a method for predicting a cardiovascular risk factor according to an embodiment of the present disclosure.

FIG. 3 is a block diagram illustrating a computing environment including a computing device suitable for use in example embodiments.

### DETAILED DESCRIPTION

Hereinafter, specific embodiments of the present disclosure will be described with reference to the drawings. The following detailed description may be provided to provide a comprehensive understanding of the methods, apparatus, and/or systems described herein. However, this is merely illustrative, and the present disclosure is not limited thereto.

In describing embodiments of the present disclosure, when it is determined that the detailed description of the known technology related to the present disclosure may unnecessarily obscure the gist of the present disclosure, the detailed description thereof will be omitted. And, terms to be described later may be defined in consideration of functions in the present disclosure, which may vary according to intentions, customs, or the like of users and operators. Therefore, the definition should be made based on the content throughout this specification. The terminology used in the detailed description may be for the purpose of describing embodiments of the present disclosure only, and should in no way be limiting. Unless explicitly used otherwise, expressions in singular include the meaning of its plurality. In this description, expressions such as “comprising,” “including,” or “having” may be intended to indicate certain features, numbers, operations, acts, elements, or a portion or a combination thereof, and should not be construed to exclude presence or possibility of one or more of other features, numbers, steps, operations, elements, or a portion or a combination thereof, in addition to those described.

Meanwhile, an embodiment of the present disclosure may include a program for performing methods described in this specification on a computer, and a computer-readable recording medium including the program. The computer-readable recording medium may include a program instruction, a local data file, a local data structure, or the like, alone or in combination. The medium may be specially designed and configured for the present disclosure, or may be commonly used in the field of computer software. Examples of the computer-readable recording medium may include a magnetic medium such as a hard disk, a floppy disk, and a magnetic tape, an optical recording medium such as a CD-ROM and a DVD, and a hardware device specially configured to store and execute a program instruction such as a ROM, a RAM, a flash memory, or the like. Examples of the program may include a high-level language code that may be executed by a computer using an interpreter or the like, as well as a machine language code such as those produced by a compiler.

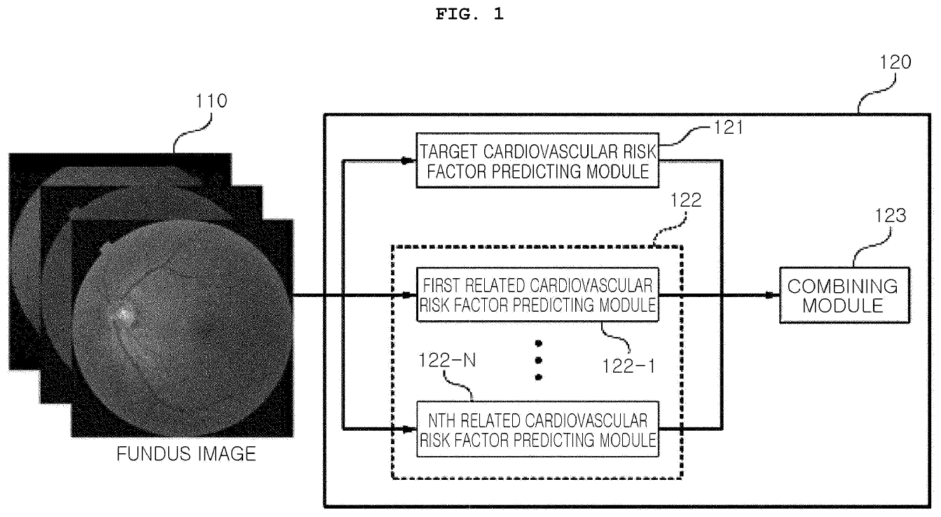

FIG. 1 is a block diagram of an apparatus 120 for predicting a cardiovascular risk factor according to an embodiment of the present disclosure.

Referring to FIG. 1, an apparatus 120 for predicting a cardiovascular risk factor according to an embodiment of the present disclosure may include a target cardiovascular risk factor predicting module 121, at least one related cardiovascular risk factor predicting modules 122, and a combination module 123.

The target cardiovascular risk factor predicting module 121 may produce an initial prediction value for a target cardiovascular risk factor (T-CRF) from a fundus image 110.

In this case, the target cardiovascular risk factor refers to a factor that may be finally predicted by the apparatus 120 among risk factors that may induce cardiovascular disease. According to an embodiment, the target cardiovascular risk factor may be, for example, a coronary artery calcification score (CACS), a carotid artery intima thickness, or the like, but is not necessarily limited to a specific factor.

According to an embodiment, the target cardiovascular risk factor predicting module 121 may produce the initial prediction value using a target cardiovascular risk factor prediction model, pre-trained using a plurality of pre-collected fundus images and an actually measured value for the target cardiovascular risk factor corresponding to each of the plurality of pre-collected fundus images.

Specifically, the target cardiovascular risk factor prediction model may be pre-trained based on a training dataset including a plurality of pre-collected fundus images and an actually measured value for the target cardiovascular risk factor corresponding to each of the plurality of pre-collected fundus images.

Also, according to an embodiment of the present disclosure, the target cardiovascular risk factor prediction model may be a convolutional neural network (CNN)-based prediction model.

The at least one related cardiovascular risk factor predicting module 122 may produce a prediction value for each of at least one related cardiovascular risk factor (R-CRF) from the fundus image 110.

In this case, the at least one related cardiovascular risk factor refer to a factor related to the target cardiovascular risk factor. According to an embodiment, the at least one related cardiovascular risk factor may include, for example, an age, a sex, smoking, a glycosylated hemoglobin level, a blood pressure, a pulse wave, a blood sugar level, a cholesterol level, a creatinine level, an insulin level, an intraocular pressure, or the like. However, the related cardiovascular risk factor may include various types of factors related to the target cardiovascular risk factor, in addition to the above-described examples. In addition, the related cardiovascular risk factor may be pre-selected using a feature selection technique, but may also be selected by a user having specialized knowledge, such as a doctor, according to embodiments.

The at least one related cardiovascular risk factor predicting module 122 may produce prediction values for different related cardiovascular risk factors, respectively, and the number of related cardiovascular risk factor predicting modules 122-1 to 122-N may be changed according to the number of related cardiovascular risk factors to be predicted.

According to an embodiment, the at least one related cardiovascular risk factor predicting module 122 may produce the prediction value using a related cardiovascular risk factor prediction model, pre-trained using a plurality of pre-collected fundus images and an actually measured value for each of the at least one related cardiovascular risk factor corresponding to each of the plurality of pre-collected fundus images.

Specifically, the related cardiovascular risk factor prediction model may be pre-trained based on a training dataset including a plurality of pre-collected fundus images and an actually measured value for each of the at least one related cardiovascular risk factor corresponding to each of the plurality of pre-collected fundus images.

Also, according to an embodiment of the present disclosure, the related cardiovascular risk factor prediction model may be a convolutional neural network-based prediction model.

The combining module 123 may produce a final prediction value for the target cardiovascular risk factor, based on the initial prediction value for the target cardiovascular risk factor and the prediction value for each of the at least one related cardiovascular risk factor.

Specifically, the combining module 123 may produce the final prediction value using a prediction result binding model, pre-trained using an initial prediction value for the target cardiovascular risk factor, a prediction value for each of the at least one related cardiovascular risk factor, and an actually measured value for the target cardiovascular risk factor, with regard to each of a plurality of pre-collected fundus images.

More specifically, the prediction result binding model may be based on a training dataset including an initial prediction value for the target cardiovascular risk factor, a prediction value for each of the at least one related cardiovascular risk factor, and an actually measured value for the target cardiovascular risk factor.

According to an embodiment of the present disclosure, the prediction result combination model may be, for example, a prediction model based on a regression analysis, such as a logistic regression model, a linear regression model, or the like, or an artificial neural network.

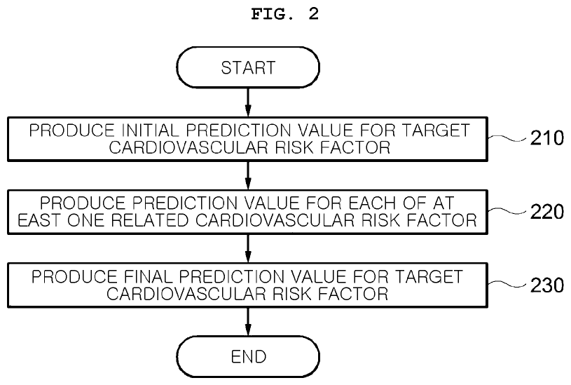

FIG. 2 is a flowchart of a method for predicting a cardiovascular risk factor according to an embodiment of the present disclosure.

A method illustrated in FIG. 2 may be performed, for example, by the apparatus 120 illustrated in FIG. 1. In a flowchart illustrated thereon, although the method is illustrated to be divided into a plurality of operations, but at least a portion of the operations may be performed in a different order, may be performed together in combination with other operations, may be omitted, or may be divided into and performed in sub-operations, or one or more operations, not illustrated, may be added thereinto and performed.

Referring to FIG. 2, first, a target cardiovascular risk factor predicting module 121 of an apparatus 120 for predicting a cardiovascular risk factor may produce an initial prediction value for a target cardiovascular risk factor from a fundus image 110 (210).

In this case, according to an embodiment, the target cardiovascular risk factor predicting module 121 may produce the initial prediction value using a target cardiovascular risk factor prediction model, pre-trained using a plurality of pre-collected fundus images and an actually measured value for the target cardiovascular risk factor corresponding to each of the plurality of pre-collected fundus images.

Specifically, the target cardiovascular risk factor prediction model may be pre-trained based on a training dataset including a plurality of pre-collected fundus images and an actually measured value for the target cardiovascular risk factor corresponding to each of the plurality of pre-collected fundus images.

According to an embodiment of the present disclosure, the target cardiovascular risk factor prediction model may be a convolutional neural network-based prediction model.

Thereafter, at least one related cardiovascular risk factor predicting module 122 of the apparatus 120 may produce a prediction value for each of at least one related cardiovascular risk factor from the fundus image 110 (220).

In this case, the number of related cardiovascular risk factor predicting modules 122 may be changed according to the number of related cardiovascular risk factors to be predicted, and related cardiovascular risk factor predicting module 122-1 to 122-N may produce prediction values for different related cardiovascular risk factors, respectively. Specifically, the at least one related cardiovascular risk factor predicting module 122 may produce the prediction value using a related cardiovascular risk factor prediction model, pre-trained using a plurality of pre-collected fundus images and an actually measured value for each of the at least one related cardiovascular risk factor corresponding to each of the plurality of pre-collected fundus images.

More specifically, the related cardiovascular risk factor prediction model may be pre-trained based on a training dataset including a plurality of pre-collected fundus images and an actually measured value for each of the at least one related cardiovascular risk factor corresponding to each of the plurality of pre-collected fundus images.

According to an embodiment of the present disclosure, the related cardiovascular risk factor prediction model may be a convolutional neural network-based prediction model.

Then, the combining module 123 may produce a final prediction value for the target cardiovascular risk factor, based on the initial prediction value for the target cardiovascular risk factor and the prediction value for each of the at least one related cardiovascular risk factor (230).

Specifically, the combining module 123 may produce the final prediction value using a prediction result binding model, pre-trained using an initial prediction value for the target cardiovascular risk factor, a prediction value for each of the at least one related cardiovascular risk factor, and an actually measured value for the target cardiovascular risk factor.

In this case, the prediction result binding model may be based on a training dataset including an initial prediction value for the target cardiovascular risk factor, a prediction value for each of the at least one related cardiovascular risk factor, and an actually measured value for the target cardiovascular risk factor.

According to an embodiment of the present disclosure, the prediction result combination model may be, for example, a prediction model based on a regression analysis, such as a logistic regression model, a linear regression model, or the like, or an artificial neural network.

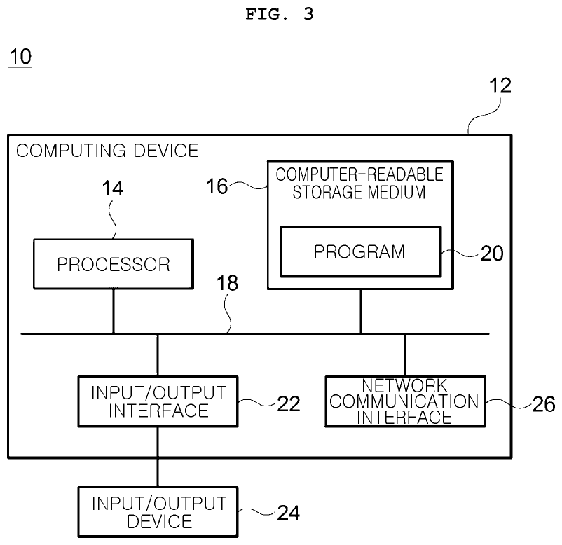

FIG. 3 is a block diagram illustrating a computing environment 10 including a computing device suitable for use in example embodiments. In an embodiment illustrated thereon, each component may have different functions and capabilities other than those described below, and may include an additional component, in addition to those described below.

A computing environment 10, illustrated, may include a computing device 12. In an embodiment, the computing device 12 may be an apparatus for predicting a cardiovascular risk factor.

The computing device 12 may include at least one processor 14, at least one computer-readable storage medium 16, and at least one communication bus 18. The processor 14 may cause the computing device 12 to operate in accordance with the example embodiments discussed above. For example, the processor 14 may execute one or more programs stored in the computer-readable storage medium 16. The one or more programs may include one or more computer-executable instructions, and the one or more computer-executable instructions may be configured for the computing device 12 to perform operations in accordance with the example embodiment, when executed by the processor 14.

The computer-readable storage medium 16 may be configured to store the computer-executable instructions, or a program code, program data, and/or other suitable form of information. A program 20 stored in the computer-readable storage medium 16 may include a set of instructions executable by the processor 14. In an embodiment, the computer-readable storage medium 16 may be a memory (a volatile memory such as a random access memory, a non-volatile memory, or a suitable combination thereof), one or more magnetic disk storage devices, optical disk storage devices, flash memory devices, other forms of storage medium accessed by the computing device 12 and capable of storing desired information, or a suitable combination thereof.

The communication bus 18 may interconnect various other components of the computing device 12, including the processor 14 and the computer-readable storage medium 16.

The computing device 12 may also include at least one input/output interface 22 and at least one network communication interface 26, providing an interface for at least one input/output device 24. The input/output interface 22 and the network communication interface 26 may be connected to the communication bus 18. The input/output device 24 may be connected to other components of the computing device 12 via the input/output interface 22. An input/output device 24, as an example, may include an input device such as a pointing device (such as a mouse, a trackpad, or the like), a keyboard, a touch input device (such as a touchpad, a touchscreen, or the like), a voice or sound input device, various types of sensor devices, and/or imaging devices, and/or an output device such as a display device, a printer, a speaker and/or a network card. The input/output device 24, as an example, may be included in the computing device 12 as a component constituting the computing device 12, and may be connected to the computing device 12 as a separate device, distinct from the computing device 12.

Although representative embodiments of the present disclosure have been described in detail above, those of ordinary skill in the art to which the present disclosure pertains will understand that various modifications are possible within the limits without departing from the scope of the present disclosure with respect to the above-described embodiments. Therefore, the scope of the present disclosure should not be limited to the described embodiments, but should be defined by the following claims as well as equivalents to the claims.

1: An apparatus for predicting a cardiovascular risk factor, the apparatus comprising:
a target cardiovascular risk factor predicting module configured to produce an initial prediction value for a target cardiovascular risk factor from a fundus image;
at least one related cardiovascular risk factor predicting module configured to produce a prediction value for each of at least one related cardiovascular risk factor from the fundus image; and
a combining module configured to produce a final prediction value for the target cardiovascular risk factor, based on the initial prediction value for the target cardiovascular risk factor and the prediction value for each of the at least one related cardiovascular risk factor.

2: The apparatus of claim 1, wherein the target cardiovascular risk factor predicting module is configured to produce the initial prediction value using a target cardiovascular risk factor prediction model, pre-trained using a plurality of pre-collected fundus images and an actually measured value for the target cardiovascular risk factor corresponding to each of the plurality of pre-collected fundus images.

3: The apparatus of claim 2, wherein the target cardiovascular risk factor prediction model is a convolutional neural network (CNN)-based prediction model.

4: The apparatus of claim 1, wherein the at least one related cardiovascular risk factor predicting module is configured to produce the prediction value using a related cardiovascular risk factor prediction model, pre-trained using a plurality of pre-collected fundus images and an actually measured value for each of the at least one related cardiovascular risk factor corresponding to each of the plurality of pre-collected fundus images.

5: The apparatus of claim 4, wherein the related cardiovascular risk factor prediction model is a convolutional neural network (CNN)-based prediction model.

6: The apparatus of claim 1, wherein the combining module is configured to produce the final prediction value using a prediction result binding model, pre-trained using an initial prediction value for the target cardiovascular risk factor, a prediction value for each of the at least one related cardiovascular risk factor, and an actually measured value for the target cardiovascular risk factor, with regard to each of a plurality of pre-collected fundus images.

7: The apparatus of claim 6, wherein the prediction result binding model is a prediction model based on one of a regression analysis or an artificial neural network.

8: The apparatus of claim 1, wherein the target cardiovascular risk factor is a coronary artery calcification score (CACS).

9: The apparatus of claim 1, wherein the target cardiovascular risk factor is a carotid artery intima thickness.

10: The apparatus of claim 1, wherein the at least one related cardiovascular risk factor comprises at least one of age, sex, smoking, a glycosylated hemoglobin level, blood pressure, a pulse wave, blood sugar level, cholesterol level, creatinine level, insulin level, or intraocular pressure.

11: A method for predicting a cardiovascular risk factor, the method comprising:
producing an initial prediction value for a target cardiovascular risk factor from a fundus image;
producing a prediction value for each of at least one related cardiovascular risk factor from the fundus image; and
producing a final prediction value for the target cardiovascular risk factor, based on the initial prediction value for the target cardiovascular risk factor and the prediction value for each of the at least one related cardiovascular risk factor.

12: The method of claim 11, wherein the producing an initial prediction value produces the initial prediction value using a target cardiovascular risk factor prediction model, pre-trained using a plurality of pre-collected fundus images and an actually measured value for the target cardiovascular risk factor corresponding to each of the plurality of pre-collected fundus images.

13: The method of claim 12, wherein the target cardiovascular risk factor prediction model is a convolutional neural network (CNN)-based prediction model.

14: The method of claim 11, wherein the producing a prediction value for each of at least one related cardiovascular risk factor produces the prediction value using a related cardiovascular risk factor prediction model, pre-trained using a plurality of pre-collected fundus images and an actually measured value for each of the at least one related cardiovascular risk factor corresponding to each of the plurality of pre-collected fundus images.

15: The method of claim 14, wherein the related cardiovascular risk factor prediction model is a convolutional neural network (CNN)-based prediction model.

16: The method of claim 11, wherein the producing a final prediction value produces the final prediction value using a prediction result binding model, pre-trained using an initial prediction value for the target cardiovascular risk factor, a prediction value for each of the at least one related cardiovascular risk factor, and an actually measured value for the target cardiovascular risk factor, with regard to each of a plurality of pre-collected fundus images.

17: The method of claim 16, wherein the prediction result binding model is a prediction model based on one of a regression analysis or an artificial neural network.

18: The method of claim 11, wherein the target cardiovascular risk factor is a coronary artery calcification score (CACS).

19: The method of claim 11, wherein the target cardiovascular risk factor is a carotid artery intima thickness.

20: The method of claim 11, wherein the at least one related cardiovascular risk factor comprises at least one of age, sex, smoking, a glycosylated hemoglobin level, blood pressure, a pulse wave, blood sugar level, cholesterol level, creatinine level, insulin level, or intraocular pressure.

21: A computer program stored in a non-transitory computer-readable storage medium, the computer program comprising at least one instruction,
wherein, when executed by a computing device having at least one processor, the computer program is configured for the computing device to:
produce an initial prediction value for a target cardiovascular risk factor from a fundus image;
produce a prediction value for each of at least one related cardiovascular risk factor from the fundus image; and
produce a final prediction value for the target cardiovascular risk factor, based on the initial prediction value for the target cardiovascular risk factor and the prediction value for each of the at least one related cardiovascular risk factor.
